(19) United States
(12) Patent Application Publication
(10) Pub. No.: US 2022/0072214 A1
(43) Pub. Date: Mar. 10, 2022

(54) Medical Instrument for Minimally Invasive Therapy, Comprising at Least Two Separate Suction Lines

(71) Applicant: W.O.M. World of Medicine GmbH, Berlin (DE)

(72) Inventor: Stan Schnüttgen, Berlin (DE)

(73) Assignee: W.O.M. World of Medicine GmbH, Berlin (DE)

(21) Appl. No.: 17/418,661

(22) PCT Filed: Dec. 27, 2019
(86) PCT No.: PCT/DE2019/000336

§ 371 Date: Jun. 25, 2021

(30) Foreign Application Priority Data
Dec. 28, 2018 (DE) 10 2018 010 008.2

(51) Int. Cl. A61M 1/00

(52) U.S. Cl. CPC A61M 1/772; A61M 1/80

(57) ABSTRACT

Disclosed is a medical instrument for use in minimally invasive therapy that generates, by at least two independent devices, an aspiration that advantageously causes the stability of the distension and the viewing conditions by adjusted flow rates.

### BACKGROUND OF THE INVENTION

### Field of the Invention

A medical instrument for use in minimally invasive surgery that includes at least two different, independently controllable suction lines. Preferably, the suction lines have different aspiration characteristics.

### Background of the Related Art

In minimally invasive surgery, frequently a cavity (a natural or artificially prepared body cavity) is distended by introducing a fluid that pressurizes the cavity and thus enables the distension. By inserted medical instruments (see below), therapeutic treatments are performed, for instance, smoothing of cartilage. As an example, such a device is shown in EP 1382291 A2. Herein, according to the intended purpose, fluid is sucked off by the respective medical instrument, in order to secure the function thereof or to minimize the side effects on the distended cavity. Examples for such medical instruments include instruments with moving blades, wherein the aspiration attracts the tissue to be separated and removes the tissue remainders from the cavity (shaver, morcellator), and carries them out of the cavity. Further examples for such medical instruments are the removal devices operated with high-frequency current (RF instruments), wherein the generated side products of the application such as water vapor or exhaustion gases are sucked off. In addition, the generated heat can be discharged by means of aspiration of the fluid by the instrument body from the cavity. In order to be able to maintain the pressure and thereby the distension of the cavity, the supply of the fluid to the cavity is correspondingly increased by an adjusted control process, that is, by at least the volume sucked off. This feedback of the sucked-off fluid is difficult, since the amounts or times of the aspiration sucked off through the medical instruments are achieved by opening valves at the handpiece by the surgeon or in a coupled manner by valves at the medical instrument and are not known, based on sensors, to the medical device for enabling fluid management.

Typically, medical devices for enabling fluid management have on the inflow side only a pressure sensor that can determine, by the fluid communication along the supply line terminating in the cavity, the pressure in the cavity and base a control process thereon.

It is disadvantageous, further, that only based on an indirectly coupled pressure sensor, a re-supply of the fluid leading, by the aspiration at the medical instruments, to a reduction of the distension, can be achieved. In order to solve this problem, there can be found, in prior art, the approach to detect an activation of the medical instrument connected to the aspiration system and to initiate a corresponding re-supply or increase of supply. Detection occurs through the current requirement (e.g., EP 2 165 720) by directly connecting the medical instrument to a supply port of the medical devices for providing fluid management. Further possibilities for detecting the operation of the medical instrument are known. A disadvantage of this solution is the high complexity for detecting the operating condition, without, however, obtaining information about the size of the leakage. Therefore, the control of the aspiration process, in order to prevent a collapse of the cavity in case of a suddenly occurring higher discharge (e.g., by using a shaver), nevertheless needs to employ further sensors or devices for this compensation, since, although the time of the outflow therethrough is known, the amount of leakage depends on the flow resistance of the used medical instruments, which normally are replaced and are not firmly connected to the control unit of the medical instruments.

Another difficulty is securing the amount of fluid flowing through the cavity with simultaneously as stable pressure as possible. The flow rate is required for removing the ablated tissue and securing the viewing conditions in the cavity.

The flow rate can be held constant and is pre-selected by the surgeon or should be provided individually or additionally upon request by the surgeon with a temporarily or permanently increased rate (flush/wash). In case that leakages through openings of the distended cavity exist, fluid will escape and the pressure will drop, so that, although the flow is secured due to the amount of the introduced fluid, in some circumstances a pressure cannot be maintained due to a too large outflow rate.

Another approach is to hold the pressure constant. Thereby, the flow rate is very large in case of leakages, since in some circumstances, the outflow resistance is very low or the cross-sectional area for the outflow is very large, once changes of the situation occur, such as additional incisions and removal of instruments.

Another situation, where such an aspiration system is used, requires a balancing of the introduced and sucked-off amount of fluid. This serves for assessing the load on the patient, whose cavity is distended, by the received fluid. For this purpose, the portions outflowing from the patient are collected on bottom layers provided with a tube connection to a vacuum vessel and funnel, same as the fluid portions from the bottom under the treatment site by a bottom aspirator, in a vacuum vessel. This vacuum vessel is connected to an aspiration system that generates negative pressure required for the transportation to the vacuum vessel. When one of the feeds is not filled with fluid—in this case, the fluid is a liquid—but is open to the atmosphere, the negative pressure will very quickly break down and other vessels also connected to this negative-pressure source, if applicable, cannot transport the fluid, since preferably over the part of lowest resistance—here between vacuum vessel and atmosphere through the transportation of air—the negative pressure is compensated. A connected medical instrument will, thus, in such a situation of a negative pressure compensated in the negative-pressure branch, not be controlled through the suction line to the vacuum vessel and not be passed to the required extent, but only through the fluid, which is driven through by the pressure of the distension. When the medical instrument is an instrument operating with moving blades, where the aspiration system attracts the tissue to be separated (shaver, morcellator), the function thereof is impaired. Then a solution is to pinch the suction line with the connection to the atmosphere or to close a valve provided therein and thus to prevent the compensation of the negative pressure, thereby, however, the balancing by the intermediate collection of the not sucked-off amount of fluid being falsified, at least until the connection is re-established. Overall, this requires higher efforts by the surgeon.

Further, the fluid pump used for aspiration is provided with specific characteristics according to its technical design. When a peristaltic pump is used, the flow characteristics (pressure and flow) are more easily controllable, and clogging of the suction lines by tissue remainders can be detected more easily and quickly. The pump device per se is smaller and produces less noise than a Venturi or diaphragm pump. The peristaltic pump can be regarded as a flow sink and is advantageous when an exact and very defined flow is required.

Advantages of the Venturi or diaphragm pump are a pressure pulse-free, uniform flow, and a very fast rise time. The Venturi or diaphragm pump is used, in the context of the invention, in minimally invasive surgery typically for generating a negative pressure by means of the transfer of gaseous fluid and requires for the aspiration a rigid negative-pressure vessel, where the sucked-off fluid is collected. Other applications are imaginable, and then the pump will, however, come into contact with the sucked-off fluid and has to be designed as a disposable product or has to be cleaned. The Venturi or diaphragm pump can be regarded as a pressure sink and is advantageous with high flow resistance. These kinds of pumps are called negative-pressure generating pump devices.

The peristaltic pump can pump the sucked-off fluid directly through the inserted tube into an open vessel, will act, however, due to its principle of operation of pinching a tube portion by rollers, also as a blocking mechanism against an imposed negative pressure. Thus, the peristaltic pump can also be connected with its transfer side to the aspiration side of another pump (e.g., the negative-pressure vessel).

A concept with two different pump devices is known from WO 93/17729. WO 93/17729 discloses a solution of coupled large and small negative-pressure vessels with respectively other connected pump devices for use as an aspiration-flushing instrument in intraocular therapy. These pump devices are used alternatively and are connected via the negative-pressure vessel to an aspiration-flushing instrument with one suction line only. The main difference to the situation this invention is based on is that only one instrument is used, as described in WO 93/17729, with different aspiration pump characteristics. The situation this invention is based on poses different requirements for the suction lines, by that they are connected to different medical instruments. In WO 93/17729 is described an aspiration-flushing instrument, the properties of which are known, and which is firmly connected to the tube cassette.

In the situation this invention is based on, according to the invention, multiple configurations of using individual suction lines are advantageous, and according to the invention, the properties of the medical instruments connected to the medical device for providing fluid management are not known in the art.

### SUMMARY

A solution according to the invention for a medical device for providing fluid management for flushing a cavity consists of a device with at least one pump device for liquid supply and two suction devices that are controllable separately from each other.

The invention, therefore, relates to a medical device for flushing cavities in minimally invasive surgery, including
(i) a storage container (1) for the flushing liquid,
(ii) a supply line (2) for supplying flushing liquid into the body cavity (11),
(iii) a controlled pump (3) for liquid supply,
(iv) a controlled first negative-pressure pump (4),
(v) a controlled second negative-pressure pump (5),
(vi) a first medical instrument (6) with a first suction line (7),
(vii) a second medical instrument (8) with a second suction line (9),
(viii) a waste container (10) connected to the first suction line (7) and the second suction line (9).

In a particular embodiment of the invention, the waste container (10) is connected by the line (12) to the first suction line (7), the second suction line (9), and the controlled second negative-pressure pump (5).

Further embodiments of the invention are described in the following.

A preferred pump device for liquid supply comprises a roller wheel pump, as described in prior art in a large number of variants. For the device according to the invention, a pump is used that can transfer a fluid flow of 2.5 l/min and can create a pressure of 300 mm Hg. Alternatively, for the purpose of liquid supply, another displacement pump can be used, such as a diaphragm pump or an impeller pump, if they can create the same fluid flow and pressure.

For the aspiration device, roller wheel pumps, Venturi pumps, or diaphragm pumps can be used, further, all kinds of reciprocating compressors, screw compressors, and turbo-compressors according to the invention.
Roller wheel pumps have already been described above with respect to prior art.
In diaphragm pumps, a separating diaphragm is mechanically moved for cyclically increasing or decreasing a chamber in size. Two valves disposed in the entry and exit of the chamber provide for that the fluid flow can only occur unidirectionally. For the purpose of the invention, the diaphragm is driven by means of a brushless DC electric motor.
In Venturi pumps (also called jet pumps), a drive jet is used to generate the pumping action. The drive jet may consist of a liquid (preferably water) or a gas (e.g., air or nitrogen). For the purpose of the invention, it is preferred to use air as the drive jet.

In any case, it is necessary that the pump used in the aspiration device can generate a negative pressure of 450 mm Hg and a liquid flow of 1.5 l/min. Roller wheel and diaphragm pumps have the advantage to be controllable very precisely. In comparison, Venturi pumps are less precise. Improvements in controllability can be obtained by valve control.

The device according to the invention may include two identical pumps for the aspiration, such as two roller wheel pumps or two Venturi pumps. Preferably, the device according to the invention includes two different pumps for the aspiration, e.g., a roller wheel pump and a Venturi pump, or a roller wheel pump and a diaphragm pump, or a Venturi pump and a diaphragm pump.

Particularly preferably, the device according to the invention includes a roller wheel pump and a Venturi pump, or a roller wheel pump and a diaphragm pump for the aspiration.

A special embodiment of the invention includes at least two roller wheels that pinch a tube, as a peristaltic pump, and at least one pump device for generating a negative pressure as a Venturi pump or a diaphragm pump. A roller wheel (inflow) is used to expose the cavity to be distended to a fluid. The inflow roller wheel is provided with a sensor for the rotational speed. A pressure sensor is coupled to the tube set via a diaphragm or a similar solution. The pressure sensor indirectly detects the cavity pressure. The rotation indirectly detects the transfer rate. A second roller wheel (outflow) may also comprise a sensor for the rotational speed and is used to suck off the fluid from the cavity with at least one tube.

A pump device for generating a negative pressure as a Venturi pump or a diaphragm pump is used to suck off fluid from the tube via a negative-pressure vessel.

This may occur simultaneously or independently with the aspiration by the outflow roller wheel.

By using a device according to the invention, in the simplest case, the constant demand of fluid flow for the flushing process (flow rate) can be achieved via a suction line, and increased demand of fluid flow for the additional flushing process can be achieved via at least one further suction line. For this purpose, in the simplest case, no valves are required, since the peristaltic pump is sealed, and the pump device generating the negative pressure can be reduced so far that nothing will be sucked off. The advantage over a pure double roller pump results from the easy generation of a high pre-pressure for aspiration by a high flow resistance and the prevention of slip at the roller wheel in case of high speeds, thereby the transfer rates will uncontrollably vary. The high speeds are necessary when a high pre-pressure needs to be generated for the aspiration. In this case, pre-pressure means the negative pressure at the pump device that is achieved by the aspiration with the negative-pressure pump. Due to the pressure differential between joint cavity and negative-pressure vessel, there results a flow that depends on the flow resistance of the used instrument.

The basic function of a flow at stable pressure in the cavity can be achieved in an embodiment by the combination of the inflow roller wheel with aspiration via the negative-pressure pump. Herein, as an input for the control process, the given pressure in the cavity is controlled as in prior art. As the input for the control, a sensor detects the fluid pressure by a diaphragm being in fluid contact with the cavity. This sensor is positioned in the fluid path behind the inflow roller wheel. A constant flow is generated by coupling this inflow roller wheel with the volume discharged by the generated negative pressure. When inflow and outflow are identical, a flow with a stable cavity pressure is obtained. When further actions by the surgeon occur, such as a request of higher flow, additional suction lines are opened. Thus, for instance, the outflow roller wheel can be used to generate a corresponding (additional) negative pressure in another suction line that is also connected to the negative-pressure vessel. This negative pressure generated by the outflow roller wheel is directly compensated by the inflow roller wheel, for instance, by increasing the rotational speed of the inflow roller wheel by the rotational speed of the outflow roller wheel. Other solutions that approximate the transferred volume flow in a better way, are also imaginable. The volume transferred per time unit (volume flow rate) can vary according to the tube diameter and thickness of the tube wall and the used material. By the direct volume increase of the inflow side in response to the activation of the outflow roller wheel, a pressure breakdown by a higher removal of fluid from the cavity is efficiently prevented.

Further embodiments with at least two suction lines as individually controllable peristaltic pumps, or with at least two suction lines with separately controllable diaphragm or Venturi pumps or other combinations also fall under the invention.

### BRIEF DESCRIPTION OF THE DRAWINGS

FIG. 1 provides an illustration of an embodiment of a medical instrument for minimally invasive therapy which includes two separate suction lines.

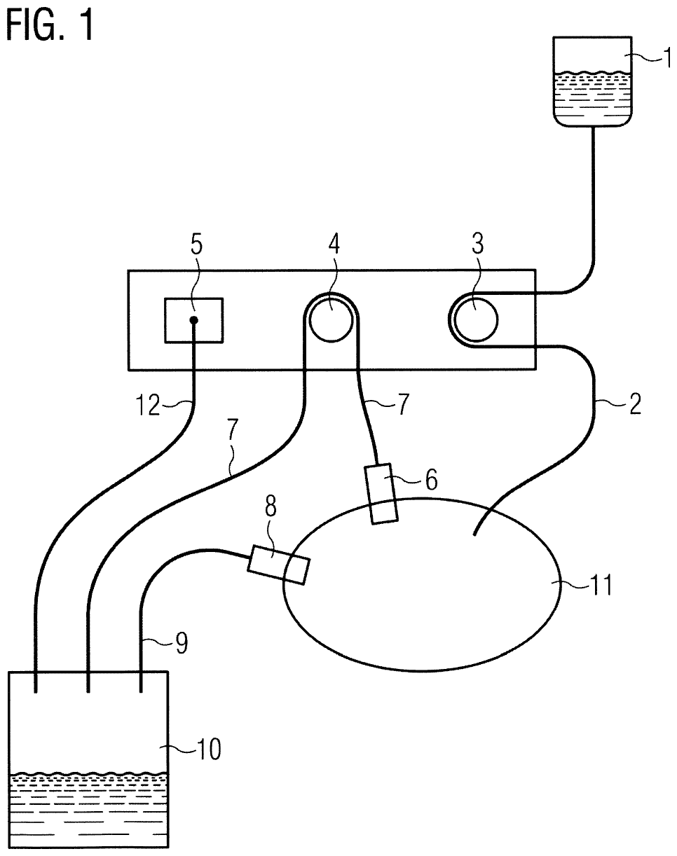

### DETAILED DESCRIPTION OF PREFERRED EMBODIMENTS

The present invention will be explained in more detail with reference to the following examples, without any limitation being intended by the examples. The person skilled in the art can develop, based on the present descriptions and his or her skills in this field, further embodiments of the invention, without inventiveness being required.

A possible configuration of the pump devices and of the cavity may be such that the inflow pump is connected via a supply line to the endoscope for viewing the cavity, and to the outflow roller wheel is connected, on the negative-pressure side, an RF instrument as well as a medical instrument with moving blades (e.g., a shaver) that can be controlled by a pinching device or a valve for the individual tube. The pump device generating a negative pressure is directly connected via the negative-pressure vessel to the cavity (third suction line, see the end of this section). The outflow roller wheel is also connected, on the positive pressure side, to the negative-pressure vessel that serves as a collection vessel and that correspondingly can be designed sufficiently large so that, during a typical procedure, there is no need to replace or empty it. The suction branch at the roller wheel is closed in operation and standstill so that the negative pressure is not affected by the operating condition of the roller wheel. The two suction lines at the outflow roller wheel can be individually occluded by pinching devices so that the following conditions
1-0 (negative-pressure pump off-outflow roller wheel stopped),
0-1 (negative-pressure pump closed-outflow roller wheel moving),
1-1 (negative-pressure pump off-outflow roller wheel moving) per pinching device and condition,
0-0 (negative-pressure pump closed-outflow roller wheel stopped), can be generated through the standstill of the roller wheel.

The condition of the third suction line can be controlled through control of the negative-pressure pump.

It is advantageous to arrange the pinching device between outflow roller wheel and medical instrument and to control it through a tripping device accessible for the surgeon. This solution enables the device to provide fluid management for detecting the condition of the pinching device and to include it in the pressure and flow control process.

Example 1: In the embodiment outlined above, when using the medical instruments (for instance, shaver or RF), an in/outflow management can be performed, without depending on the detection of the operating conditions of the medical instruments. The outflow roller wheel pump and the pump device generating a negative pressure provided in the design concept of the device for providing fluid management can provide separately controllably the respectively required negative pressures and flow rates for the suction lines, i.e., the fluid connections of the cavity to the device. Both pump devices are controllable, the outflow roller wheel being able, for instance, to accept a higher aspiration of the medical instrument (e.g., shaver/RF), and the pump device generating a negative pressure securing a continuous flow. It is a big advantage for the surgeon that changing the connections or closing the cock at the trocar, which is the typical sleeve used for introducing the endoscope, can be dropped.

In the scenario of arthroscopy, in this example, fast pressure changes are avoided, since in prior art solutions, high demand of re-supply will occur, when the suction lines to a medical instrument (e.g., shaver/RF) are open, by the occurring pressure drop at the sensor at the inflow roller wheel, and this can only relatively late be compensated, whereby due to the pressure peaks, larger extravasation of the fluid will result, than with the solution according to the invention. Extravasation leads to disadvantageous swellings in the environment of the distended cavity. This embodiment of the solution according to the invention allows for the first time a “low-pressure arthroscopy” with slightly more than 30 mm Hg cavity pressure in the joint, since with the prior art devices the pressure variations are too large and the cavity will collapse.

Example 2: In the embodiment outlined above, a medical instrument with a high demand of flow rate (e.g., a shaver, morcellator, or other devices that separate and/or suck off tissue pieces) can directly be connected at the outflow roller wheel that transfers into the negative-pressure vessel, and a medical instrument with high flow resistance (e.g., an RF instrument) can directly be connected at the pump device generating a negative pressure and via the same suction line at the cavity. The medical instrument with high flow resistance is disconnected through a valve, and only upon activation, the suction line is opened there.

The advantage is a fast aspiration effect at the medical instrument with high flow resistance at a very stable cavity pressure and a flow that is adjustable independently on the cavity pressure. In addition, this example has the advantage for the surgeon that changing the connections or closing the cock at the trocar can be dropped.

Example 3: In the embodiment outlined above, the outflow roller wheel can directly be connected to the cavity, and the pump device generating a negative pressure can be connected to a medical instrument with moving blades (e.g., shaver, morcellator or other devices that separate and/or suck off tissue pieces). Thus, the cavity pressure can easily be monitored, and the in- and outflow can adequately be controlled. When the medical instrument with moving blades is activated, there will be, due to the pumping principle, no pressure variations, and a stable cavity wall results thereby. This is particularly helpful in cavities with very flexible walls such as a uterus.

Example 4: In the embodiment outlined above, the outflow roller wheel and the inflow roller wheel can directly be connected to an endoscope with separated channels for in- and outflow, that endoscope being located in the cavity, for generating a continuous flow. The pump unit generating a negative pressure can be connected to an aspiration instrument or additionally via a valve in a removable manner to the endoscope. In urology, for stone removal in the region of the ureters or the renal calyx, the stone is fixed with an instrument that grips around the stone (stone extractor basket). The procedure of gripping around can be promoted by activation of the suction line with an additional aspiration pulse created by the pump unit generating a negative pressure, so that by the additional aspiration process, the stone is positioned in the instrument. This pulse-like aspiration increase is very advantageous, since the situation in the cavity is not changed, and only an additional suction in the direction of the aspiration opening is generated, the effect of which can be compensated by an increased inflow. Due to the length and diameter of the tube (i.e., flow resistance), an enormous pre-pressure is required that can best be generated with the pumping principle of the pump unit generating the negative pressure. In normal operation, an exact pressure control over the smallest amount is required (risk of damage to the renal calyx region), which can best be achieved with peristaltic pumps, however, they cannot generate quickly enough the pre-pressure required for a suction pulse, or would be by far overbuilt if they did.

### LIST OF REFERENCES

(1) storage container for the flushing liquid,
(2) supply line for supplying flushing liquid into the body cavity (11),
(3) controlled pump for liquid supply,
(4) controlled first negative-pressure pump,
(5) controlled second negative-pressure pump,
(6) first medical instrument,
(7) first suction line at first medical instrument,
(8) second medical instrument,
(9) second suction line at second medical instrument,
(10) waste container,
(11) body cavity,
(12) line from the waste container to the controlled second negative-pressure pump.

1. A medical instrument for flushing cavities in minimally invasive surgery, comprising
(i) a storage container (1) for the flushing liquid,
(ii) a supply line (2) for supplying flushing liquid into the body cavity (11),
(iii) a controlled pump (3) for liquid supply,
(iv) a controlled first negative-pressure pump (4),
(v) a controlled second negative-pressure pump (5),
(vi) a first medical instrument (6) with a first suction line (7),
(vii) a second medical instrument (8) with a second suction line (9),
(viii) a waste container (10) connected to the first suction line (7) and the second suction line (9).

2. The medical instrument of claim 1, characterized by that the waste container (10) is connected by the line (12) to the first suction line (7), the second suction line (9), and the controlled second negative-pressure pump (5).

3. The medical instrument of claim 1 or 2, characterized by that the controlled pump (3) for liquid supply is a roller wheel pump.

4. The medical instrument of claim 1, 2, or 3, characterized by that the controlled first negative-pressure pump (4) and/or the controlled second negative-pressure pump (5) is a roller wheel pump.

5. The medical instrument of a least one of claims 1 to 4, characterized by that the controlled first negative-pressure pump (4) and/or the controlled second negative-pressure pump (5) are different.
